(19) United States
(12) Patent Application Publication
(10) Pub. No.: US 2008/0112845 A1
(43) Pub. Date: May 15, 2008

(54) Air Cleaning Unit, and Method of Air Disinfection

(76) Inventors: Charles E. Dunn, Memphis, TN (US); Charles E. Dunn, Hughes, AR (US)

Correspondence Address: BAKER, DONELSON, BEARMAN, CALDWELL & BERKOWITZ, PC, 900 SOUTH GAY STREET, 2200 RIVERVIEW TOWER, KNOXVILLE, TN, 37902, US

(21) Appl. No.: 11/939,534

(22) Filed: Nov. 13, 2007

Related U.S. Application Data
(60) Provisional application No. 60/859,197, filed on Nov. 15, 2006.

(51) Int. Cl. A61L 2/10; A61L 2/00; F21V 23/00; F24F 3/16

(52) U.S. Cl. 422/24; 362/221; 422/121

(57) ABSTRACT

An air cleaning unit is provided. The air cleaning unit includes a housing having an air intake and an air outlet. Intermediate the air intake and air outlet is an air filtration medium. Also within the housing is at least one UV-C lamp. The air cleaning unit also includes an odor-reducing medium, such as a photocatalytic oxidation (“PCO”). filter. A method for filtering air using the air cleaning unit is also provided.

### STATEMENT OF RELATED APPLICATIONS

This application claims priority to a pending provisional application having U.S. Ser. No. 60/859,197. That application is titled “Air Cleaning System, and Method of Air Disinfection.” That application was filed on Nov. 15, 2006, and is incorporated herein in its entirety by reference.

### NOTICE OF COPYRIGHTS AND TRADE DRESS

The textual description of the inventions in this patent document contains material which is subject to copyright protection. The copyright owner has no objection to the facsimile reproduction of the patent disclosure as it appears in the Patent and Trademark Office patent files or records, but otherwise reserves all rights under the Copyright Act as found at 17 U.S.C. § 101, et. seq.

### BACKGROUND OF THE INVENTION

1. Field of the Invention

The present invention generally relates to air filtration. More particularly, the present invention relates to an air cleaning unit. The present invention also relates to methods of air disinfection and purification.

2. Description of the Related Art

It is known to use ultraviolet light to control airborne pathogens. Ultraviolet light is radiation having a wavelength of about 400 nanometers or less. One type of ultraviolet light is “UV-C” light. UV-C light is a high frequency wavelength of light within the ultraviolet band. Certain UV-C light has a wavelength of between 100 and 280 nm and has been shown to have bactericidal effects. In this respect, ultraviolet (UV) rays within the UV-C wavelength can destroy pathogens such as viruses, yeast, bacteria, mold, and mildew by breaking through the outer membrane of the airborne pathogens. When the radiation reaches the DNA of the pathogen, it causes modifications. The DNA then transmits incorrect codes, rendering the pathogen unable to reproduce.

UV light may be produced artificially by electric-arc lamps. Ultraviolet light may also be generated by mercury bulbs. Such light sources are sometimes referred to as ultraviolet germicidal lamps. Ultraviolet germicidal lamps provide a more powerful and concentrated effect of UV energy than can be found naturally.

The availability of low to medium pressure mercury bulbs that generate UV-C light has led to the development of devices used for the disinfection and sterilization of objects. An example is the use of UV light in barber shops for the sterilization of combs and cutting instruments. U.S. Pat. No. 6,656,424 discloses a method for sterilizing hospital or surgical areas using ultraviolet radiation. U.S. Pat. App. No. 2004/0025899 shows a canister that receives toothbrushes. The canister includes an internal ultraviolet bulb for sanitizing toothbrushes once they are received into the canister. U.S. Pat. No. 6,627,000 teaches the use of UV-C light for the disinfection of surfaces such as fins and coils in a heating, ventilating, and cooling (HVAC) system. This patent issued to Steril-Aire USA, Inc. (now Steril-Aire, Inc.) currently of Burbank, Calif.

Recently, air cleaning systems have been disclosed which use UV-C lamps. U.S. Pat. No. 6,855,295 provides a UV air cleaning and disinfecting system. The system employs an array of UV lamps and a pair of filters at an intake chamber. U.S. Pat. No. 6,497,840 teaches an air filtration apparatus that includes a UV light sterilization chamber for destroying airborne pathogenic bacteria. Air is drawn through a filter and into a sterilization chamber that is irradiated with ultraviolet light. U.S. Pat. No. 5,894,130 provides an ultraviolet sterilization unit having a housing that may be attached to an air heating and cooling system. The unit includes two openings into which lamp cartridges are inserted.

One company that has been instrumental in the development of combined air filtration and ultraviolet germicidal air disinfection technology is Lumalier Corporation. of Memphis, Tenn. This company operates under the trade name LUMALIER®. LUMALIER® offers various ultraviolet germicidal air filtration products designed to benefit the healthcare, educational, institutional, office and residential markets.

A need exists for improved air cleaning units. Further, a need exists for a unique air sterilization unit that combines, in one embodiment, air filtration, VOC removal, odor removal and UV-C disinfection.

### SUMMARY OF THE INVENTION

An air cleaning unit is provided. In one aspect, the air cleaning unit includes a housing having an air intake and an air outlet. In one aspect, the housing has at least two sides having double walls. Alternatively, the walls are insulated with a ceramic coating.

The air cleaning unit further includes a particle filtration medium intermediate the air intake and the air outlet. The particle filtration medium is preferably fabricated from a material that is resistant to deterioration under exposure to ultraviolet radiation. The filtration medium may be, for example, a 5-inch MERV-11 filter.

The unit also has at least one UV-C lamp intermediate the air intake and air outlet, along with an odor-reducing medium. Preferably, the odor-reducing medium comprises a photocatalytic oxidation filter. Such a filter is capable of filtering or absorbing volatile organic compounds. The photocatalytic oxidation filter may contain titanium dioxide.

In one embodiment, the UV-C lamps are supported by a ballast. The ballast may contain a microprocessor and, optionally, at least one specular aluminum reflector below each lamp.

A method of filtering air is also provided. In one step, at least one air cleaning unit is provided. The air cleaning unit includes a housing having an air intake and an air outlet, an air filtration medium intermediate the air intake and air outlet, at least one UV-C lamp intermediate the air intake and air outlet, and an odor-filtering medium. Preferably, the odor-filtering medium is a photocatalytic oxidation filter. In another step, an individual installs the housing of the air cleaning unit within an air moving system. The air moving system may be, for example, an HVAC system, a furnace or a motorized air filtering machine.

The housing may be installed in any orientation, such as a vertical or a horizontal orientation. In the case of an air handling system, the air intake receives air from the return air ductwork, and the air outlet distributes cleaned air to the exit ductwork of the air handling system. When the air handling system is an air conditioning or HVAC system, the air cleaning unit may be positioned adjacent to fins of the air handling system.

In one embodiment, at least one UV-C lamp is adjacent the air intake. In an alternative embodiment, at least one UV-C lamp is oriented perpendicular to the fins of an HVAC system. In one aspect, a plurality of air cleaning units are provided in parallel in order to pull and simultaneously filter air as it enters ductwork of an air conditioning system.

A method of bidding on a job specification is also provided. One step comprises receiving a request for a bid for the installation of an air cleaning unit from a potential customer. Receiving a request may include soliciting a request or invitation to bid. Another step includes responding to the bid by offering an air cleaning unit comprising a housing having an air intake and an air outlet, an air filtration medium intermediate the air intake and air outlet, at least one UV-C lamp intermediate the air intake and air outlet, and a photocatalytic oxidation filter. The method may further comprise the step of installing the air cleaning unit into an air handling system for the potential customer.

### BRIEF DESCRIPTION OF THE DRAWINGS

So that the above recited features of the present inventions can be better understood, certain drawings or flow charts are appended hereto. It is to be noted, however, that the appended drawings illustrate only selected embodiments of the inventions and are therefore not to be considered limiting of scope, for the inventions may admit to other equally effective embodiments and applications.

FIG. 1 presents a perspective view of an air cleaning unit of the present invention, in one embodiment.

FIG. 2 is a bottom view of the system of FIG. 1. In this view, a bottom side of the housing is visible representing an air intake.

FIG. 3 is a top view of the system of FIG. 1. In this view, a top side of the housing is visible representing an air outlet.

FIG. 4 is a front view of the housing of FIG. 1.

FIG. 5 is a side view of the system of FIG. 1. In this view, the right side of the housing is visible.

FIG. 6 is a back view of the housing of FIG. 1.

FIG. 7 is another side view of the system of FIG. 1. In this view, the left side of the housing is visible.

FIG. 8 presents an exploded view of certain cleaning components of the air cleaning unit of FIG. 1.

FIGS. 9A and 9B present perspective views of the air filtration medium used in the air cleaning unit of FIG. 1, in one embodiment.

In FIG. 9A, the filtration medium is shown facing up.

In FIG. 9B, the filtration medium is shown facing down.

FIGS. 10A and 10B show perspective views of UV-C lamps that may be used in the air cleaning unit of FIG. 1, in one embodiment.

FIG. 10A is a rear view of the UV-C lamps, with a socket being seen.

FIG. 10B is a front view of the UV-C lamps.

FIG. 11 provides a perspective view of a VOC and odor-filtering medium as may be used in the air cleaning unit of FIG. 1, in one embodiment. In this embodiment, the medium is pleated to increase surface area.

FIG. 12 is a front view of a plurality of air cleaning units assembled to receive and filter air in parallel.

### DETAILED DESCRIPTION

### Definitions

As used herein, the term “MERV” refers to the Minimum Efficiency Reporting Value, which is a rating of the effectiveness of air filters in removing particle contaminants from the air. The MERV value is dependent upon the particle size and the efficiency of the filter in capturing particles of a particular size. The MERV rating system is defined by ASHRAE (American Society of Heating, Refrigeration, and Air Conditioning Engineers), Standard number 52.2.

The term “air handler” means any system for moving air within a structure. Non-limiting examples include a heating system for a building, a cooling system for a building, or a combined “heating, ventilation and cooling system,” or “HVAC.” Air handlers may be used in either residential, office, retail or commercial structures. Air handlers typically, though not always, force air through ductwork.

The term “furnace” refers to a type of heating system that typically uses gas, oil, or electricity to heat air that is then distributed by ductwork.

### DESCRIPTION OF SPECIFIC EMBODIMENTS

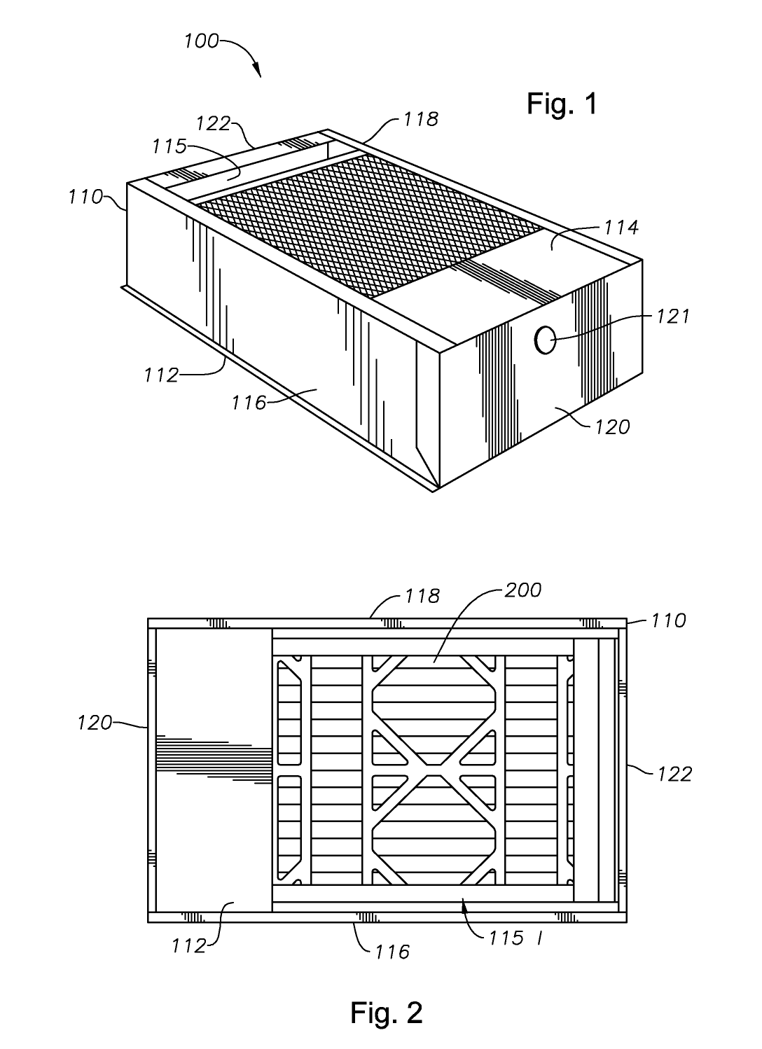

FIG. 1 presents a perspective view of an air cleaning unit 100 of the present invention, in one embodiment. The air cleaning unit 100 first includes a housing 110. The illustrative housing 110 defines a six-sided box that forms an inner chamber. The six sides are denoted as 112, 114, 116, 118, 120, and 122. The sides 112, 114, 116, 118, 120, 122 serve to contain internal air cleaning components as will be described further below.

In the view of FIG. 1, the air cleaning unit 100 is positioned such that side 112 is on the bottom, and side 114 is on the top. This is a horizontal positioning for the system 100. However, the air cleaning unit 100 may be deployed in a vertical position such that sides 112 and 114 are upright. Alternatively, sides 116 and 118 may serve as the bottom and top sides, respectively. What matters is that side 112 serves as the air intake side, and side 114 serves as the air outlet side.

FIG. 2 is a bottom view of the housing 110 of FIG. 1. In this view, side 112 is visible. As noted, side 112 serves as an air-intake side. Side 112 includes an opening 115I. Opening 115I allows air to move through side 112 and into the internal chamber of the housing 110.

An air filtration medium 200 is in FIG. 2. The air filtration medium 200 serves to filter particles as air is moved through the air-intake 115I and into the air cleaning unit 100. The air filtration medium 200 may be referred to as a particle filtration medium.

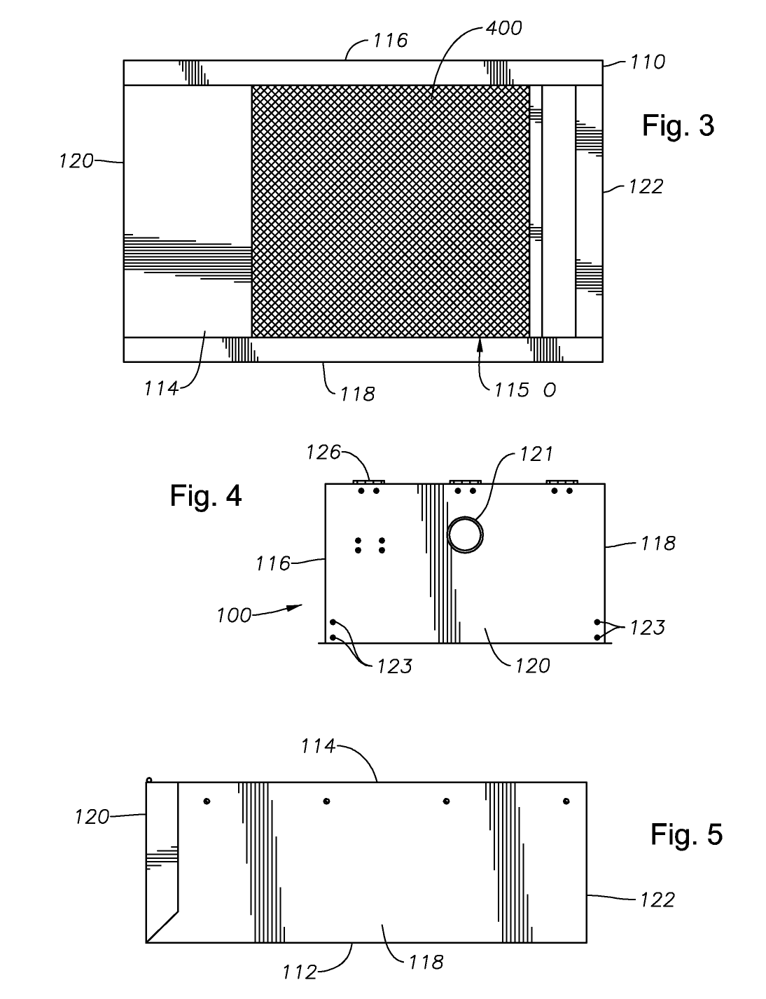

FIG. 3 is a top view of the housing 110 of FIG. 1. In this view, side 114 is visible. As noted, side 114 serves as an air outlet side. Side 114 includes an opening 115O. Opening 115O allows air to move through side 114 and out of the chamber of the housing 110.

An odor-reducing medium 400 is visible in FIG. 3. The odor-reducing medium 400 serves to at least reduce, if not eliminate, odors from air moving through filter particles as air is moved through the air cleaning unit 100. The odor-reducing medium 400 also removes volatile organic compounds, or VOC's. Certain features of the odor-reducing medium 400 are described in greater detail below.

As noted, the housing 110 also includes sides 116 and 118. Sides 116 and 118 are preferably solid side walls. In one embodiment, each side wall 116, 118 is a double-sided wall having an air chamber formed therebetween. The air chamber configuration serves an insulative purpose and also prevents sweating on the outside of the housing 110. In yet another embodiment, each side wall 116, 118 is covered with a ceramic-based paint, thus optionally reducing the need for a double-sided wall construction. In one embodiment, one or more of the sides 112, 114, 116, 118, 120, and 122 are painted with an insulative material that cures in order to provide insulative qualities to the housing 110.

Finally, front 120 and back 122 walls are provided for the housing 110. Side 120 serves as a front wall and door. In one aspect, side 120 hingeably attaches to side 114. In another aspect, front side 120 hingeably attaches to opposing side walls 116 and 118. In either embodiment, side 120 is capable of rotating from a closed position, as illustrated in FIG. 1 to an open position, as illustrated in FIG. 8. Alternatively, side 120 is completely removable. Side 120 facilitates access to the inner chamber of the housing 110 and the unit 100's air cleaning components 200, 300, 400.

FIG. 4 is a front view of the housing 110 of FIG. 1. Side 120 is fully visible in this view. As illustrated, side 120 contains a pair of door latches 123 for securing side 120 to the housing 110. Hinges 126 are also provided to allow access into the housing 110.

FIG. 5 is a side view of the housing 110 of FIG. 1. Side 118 is visible in this view. The length of side 118 may vary depending on whether the corresponding unit is a furnace or an air handler.

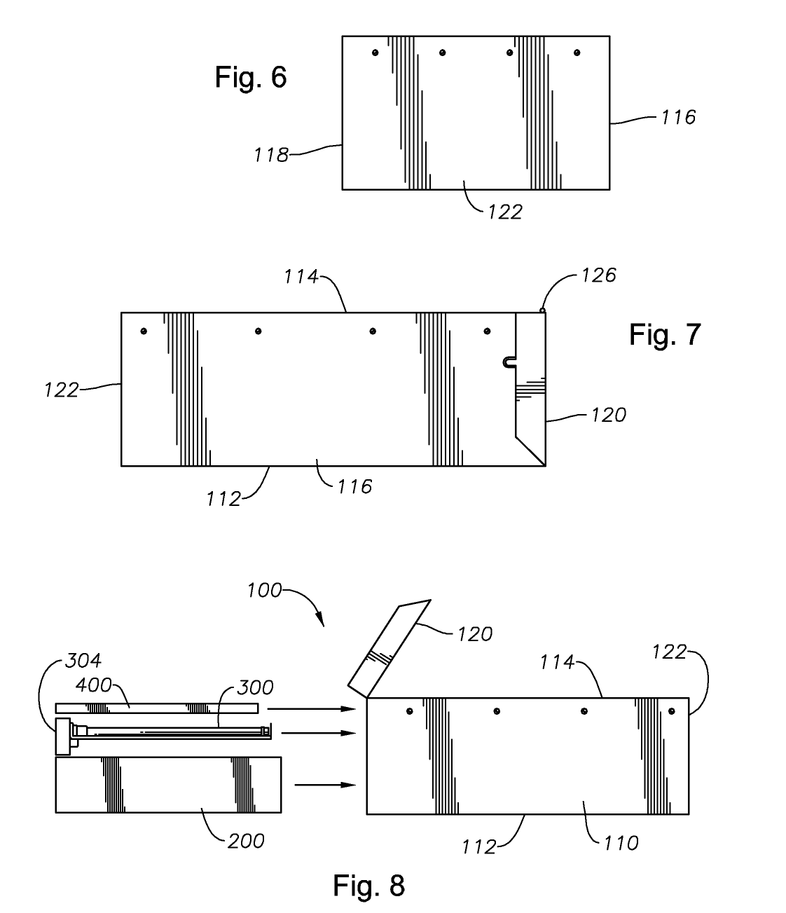

FIG. 6 is a back view of the housing 110 of FIG. 1. In this view, back side 122 is seen. Side 122 serves as a back wall. Side 122 is a solid wall and is preferably fixed to the housing 110.

FIG. 7 is a side view of the housing 110 of FIG. 1. Side 116 is visible in this view.

It is preferred that the sides 112, 114, 116, 118, 120, and 122 of the housing 110 be fabricated from a lightweight but durable metal material such as aluminum alloy or steel. In one example, the housing 110 is fabricated from a 17-gauge metal composite of aluminum and steel. The housing 110 may be dimensioned and configured to be installed into or attached within a known air handler or furnace unit having a 120V through 250V operation. In one embodiment, the housing 110 is adapted to fit and is capable of supporting the weight of a commercial or residential HVAC system such as those available from Rheem, Ruud, Weatherguard, Trane, Carrier, or Lennox. The housing 110 may be mounted either vertically or horizontally depending on the orientation of the corresponding HVAC system. In an alternative embodiment, the housing 110 is adapted to fit an in-room air cleaning device that contains no heating or cooling mechanism.

In one embodiment, the housing 110 is approximately ten inches in height. The overall width of the housing 110 may vary depending on the capacity and type of the corresponding HVAC or other air handling system. For HVAC systems having a load capacity of 1.5, 2, 2.5 or 3 tons, the front wall may be 17.5 inches. For other HVAC systems having a load capacity of 3 tons or systems having a capacity of 3.5 or 4 tons, the front wall may be about 21 inches. For HVAC systems having a load capacity of 4 or even 5 tons, the front wall may be about 24.5 inches. In a Lumalier IAQ-1 air handler unit, the side walls of the housing 110 are 21.75 inches in length. In a Lumalier IAQ-1-F Furnace unit, the side walls of the housing are 28 inches in length.

FIG. 8 presents an exploded view of three air-cleaning components 200, 300, 400 as may be used in the air cleaning unit of FIG. 1. Each of these components resides within the housing 110 of FIG. 1. However, the components 200, 300, 400 are shown exploded from the housing 110 for illustrative purposes. The components are a particle filtering medium 200, one or more UV-C lamps 300, and an odor-removing medium 400. Side 120 is shown in an open position to allow removal and service of the air-cleaning components 200, 300, 400. Side 120 is hingeably connected to side 114 allowing side 120 to function as a door.

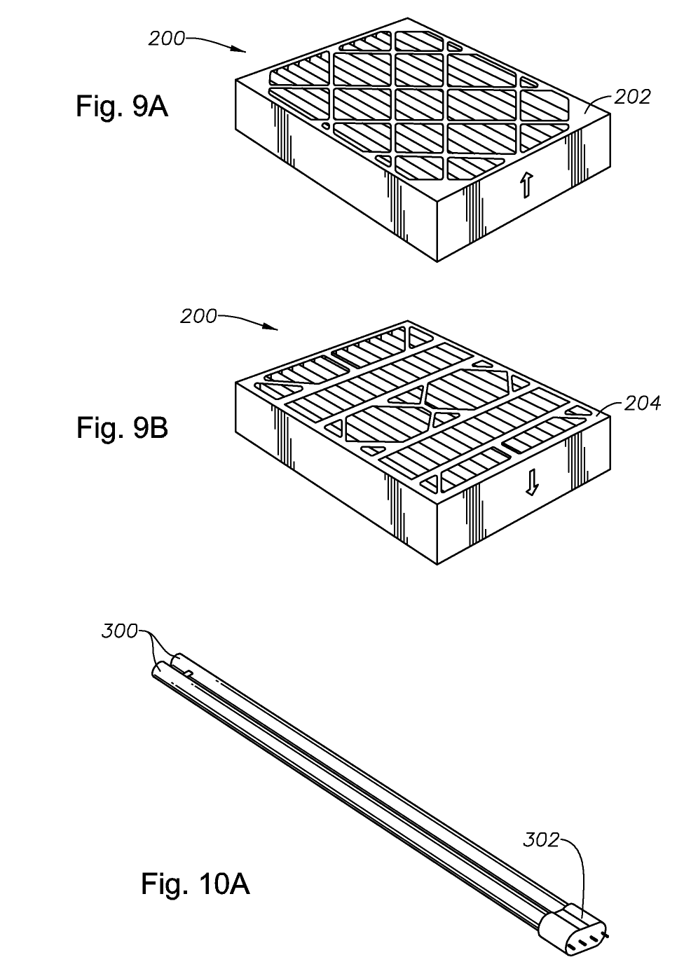

FIGS. 9A and 9B present perspective views of the particle filtering medium 200 used in the air cleaning unit 100 of FIG. 1, in one embodiment. In FIG. 9A, the filtration medium 200 is shown facing up. In FIG. 9B, the filtration medium 200 is shown facing down.

The particle filtering medium 200 has a top side 202 and a bottom side 204. When in use, air enters the opening 115I of the housing 110 and flows through the bottom side 204 of the particle filtering medium 200 before moving past one or more UV-C lamps 300 and exiting through an odor-removing medium 400.

The particle filtering medium 200 is capable of capturing various size particles in the air as it passes through the air cleaning unit 100. The average particle size efficiency is dependent upon the MERV rating of the particle filtering medium 200. Preferably, the air cleaning unit is designed to use filters rated from MERV 8 to MERV 11. Filters having various MERV ratings may be employed in the air cleaning unit 100. In one embodiment, a MERV-11 filter is used. The MERV-11 filter 200 traps mold, dust, pet dander, dust mites and other allergens.

A higher MERV rating results in increased efficiency in capturing air particles. In one example, the particle filtering medium 200 is a MERV 11, meaning the filtering medium 200 contains an average particle size efficiency of approximately 65-79.9% for particles having a size in the range of 1.0 to 3.0 microns.

In a preferred embodiment, the particle filtering medium 200 contains pleats. Pleats add surface area to the particle filtering medium 200 and reduce static pressure. This allows air to pass through at a static pressure that coincides with the capacity of the fan motor within the HVAC unit. The illustrative particle filtering medium 200 from FIGS. 9A and 9B is a pleated filter having a thickness of approximately 5 inches. However, an un-pleated filter may also be used.

In one embodiment, the particle filtering medium 200 is comprised primarily of bicomponent polyethylene/polypropylene fibers. The fibers may be treated with a UV stabilizer to prevent deterioration of the fibers. The UV stabilizer may be via introduction of carbon, ink or die.

Preferably, the particle filtering medium 200 is capable of withstanding temperatures of up to 200° Fahrenheit. Such filter mediums are available from Kimberly Clark of Hendersonville, N.C. and Neenah, Wis. A similar suitable filter medium is available from Ahlstrom Air Media of Darlington, S.C.

Filter sizes of various widths and depths may be accommodated for the filtering medium 200, depending on the dimensions of the air path within the housing 110 and the type of HVAC or other air handling system. In one embodiment, the housing 110 is adapted to receive a filtering medium 200 having a width of 15.875 inches and a depth of 19.625 inches. These dimensions correspond to one footprint of a residential or small commercial air handler or furnace. In an alternative embodiment, the width may be 19.250 inches or 22.875 inches with a depth of 19.625 inches depending on the overall capacity of the HVAC or other air handling unit.

As an alternative, the air filtration medium 200 may be a high efficiency particulate arrester (“HEPA”) filter. Alternatively, or in addition, the air filtration medium 200 may comprise an ultra-low penetration (“ULPA”) filter.

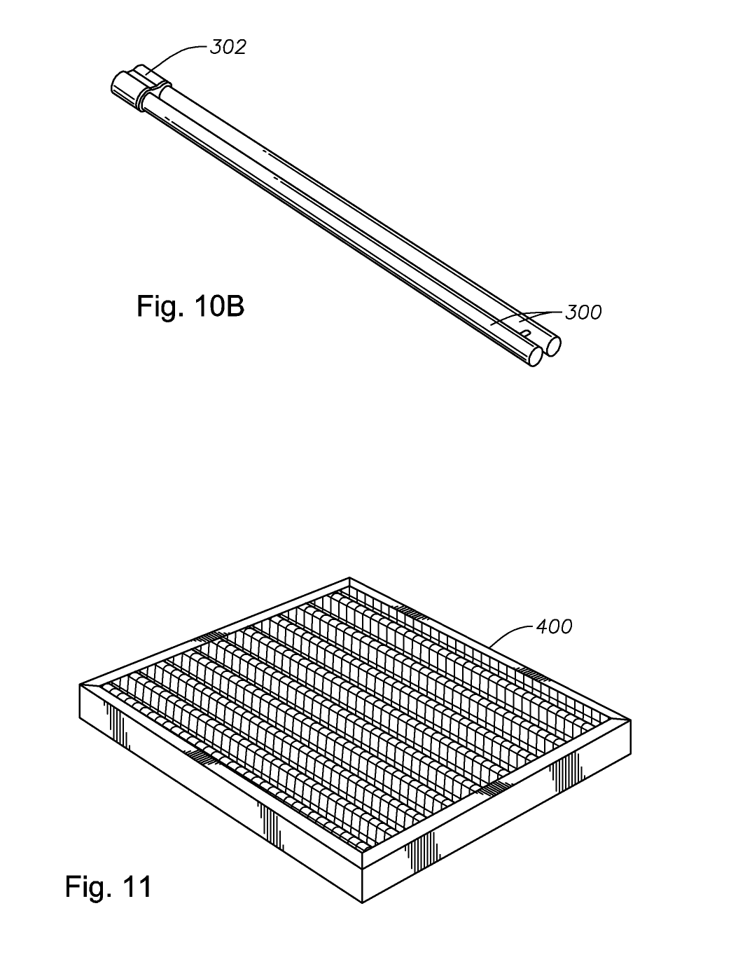

FIGS. 10A and 10B show perspective views of illustrative UV-C lamps 300 as may be used in the air cleaning unit of FIG. 1. FIG. 10A is a rear view of the UV-C lamps 300, with a socket 302 being seen. FIG. 10B is a front view of the UV-C lamps 300.

In the illustrated embodiment, the air cleaning unit 100 utilizes two PL-L high output germicidal Sterilamps® 300 available from Royal Philips Electronics of the Netherlands. Each of these lamps is 17 inches in length, has no more than 4.5 mg of mercury content, and operates in the range of 253.7 nanometers. The wattage may vary from 5 nominal watts/1.5 UV watts produced to 100 nominal watts/30 UV watts produced. Specific models suitable for operation in the cleaning unit 100 include PL-L36 WTUV and PL-L60 WTUV. Each of these models is capable of operation with electronic ballasts with lamp currents ranging from 800 mA to 1000 mA. Other suitable UV-C germicidal lamps are manufactured by General Electric and Sylvania.

In one embodiment, the air cleaning unit 100 utilizes a PureVOLT™ electronic ballast 304 (seen in FIG. 8) to operate the germicidal lamps 300. The PureVOLT™ ballast 304 is manufactured by Advance Transformer Company of Rosemont, Ill. The ballast may be microprocessor controlled and may operate at any input voltage from 120 to 277 volts and 50/60 Hz. The germicidal lamps, ballast or a combination thereof are capable of movement within the housing 110 to allow for greater UV-C contact with targeted airborne pathogens. The ballast assembly included the ballast 304, wiring (not shown), and socket 302 for holding at least two lamps. Specular aluminum reflectors (not shown) may be installed below each lamp.

In another aspect, two different wavelengths are employed in two different lamps. One lamp has a wavelength of 254 nm and is used for purification of the air. The other lamp has a wavelength of 185 nm and is used to decompose organic molecules in the air.

An internal and external safety limit switch may also be installed in each unit. The switches allow for disruption of power to the UV lamps when the unit is opened for service. Specifically, the internal switch does not allow the UV assembly to operate when removed from the cabinet. The external limit switch does not allow the unit to operate when side 120 is opened. Service technicians can activate the unit by manually depressing the external limit switch with the door open and check for proper operation of the lamps without risk of exposure to UV energy.

As air passes through the housing 110 and over the UV-C lamps 300, the UV-C lamps 300 simultaneously emit the UV-C component of ultraviolet light that destroys bacteria, yeasts, mould spores, viruses and other biological contaminants in the air. Preferably, the UV-C lamps 300 emit energy at a wavelength of about 253.7 nanometers.

The primary target for the UV-C lamps 300 in the air cleaning unit 100 is bacteria.

### Examples of Bacteria Include:

Bacillus anthracis

B. suptilis (and B. suptilis spores)

B. parathyphosus

B. suptilis (and B. suptilis spores)

Campylobacter jejuni

Clostridium tetani

Corynebaterium diphteriae

Dysentery bacilli

Eberthella typhosa

Escherichia Coli (also known as E. coli)

Klebsiella terrifani

Legionella pneumophila

Micrococcus candidus

Micrococcus sphaeroides

Mycobacterium tuberculosis

Neisseria catarrhalis

Phytomonas tumefaciens

Pseudomonas aeruginosa

Pseudomonas fluorescens

Proteus vulgaris

Salmonella enteritidis

Salmonella paratyphi

Saomonella typhimurium

Sarcina lutea

Seratia marcescens

Shigella paradysenteriae

Shigella sonnei

Spirillum rubrum

Staphylococcus albus

Staphylococcus aureus

Streptococcus faecalis

Streprtococcus hemoluticus

Streptococcus lactus

Streptococcus viridans

Sentertidis

Bibrio chlolerae (V. comma)

Yersinia enterocolitica

For such bacteria, an ultraviolet dosage of about 10.0 to 100.0 J/m² at 254 nm radiation wavelength is capable of killing 90% of the bacteria. The only exception is Sarcina lutea, which generally requires a dosage of about 197.0 J/m2 at 254 nm radiation wavelength for a 90% kill rate.

Viruses are also a target of the germicidal ultraviolet lights 300. Examples of viruses include:

Hepatitis A

Influenza virus

MS-2 Coliphase

Polio virus

Rotavirus

For such viruses, an ultraviolet dosage of about 70.0 to 100.0 J/m² at 254 nm radiation wavelength is capable of killing 90% of the virus. The only exception is MS-2 Coliphase, which generally requires a dosage of about 186.0 J/m2 at 254 nm radiation wavelength for a 90% kill rate.

It is noted that mould spores have a wide range of required ultraviolet dosage for killing 90% of the spores. At a lower end, Oospora lactis only requires an ultraviolet dosage of about 50.0 J/m² at 254 nm radiation wavelength for killing 90% of the spores. At a higher end, Aspergillis niger requires an ultraviolet dosage of about 1,320.0 J/m² at 254 nm radiation wavelength for killing 90% of the spores. Several other spores generally require dosages in excess of 400 J/m² at 254 nm radiation wavelength. However, there is no need to achieve levels to disinfect or kill mould spores at such high dosage levels as the 5 inch pleated MERV filter 200 has the capacity to remove mould spores at a high level. Mould spores typically fall into the 3.0 to 10.0 micron range; as such, they are readily removed with a MERV 8 to MERV 11 filter.

It is also noted that there is an additional disinfection rate for viral and bacterial contaminate through the use of the UV energy in conjunction with odor-reducing medium 400. More specifically, the disinfection rate is increased when St. Gobain Quartzel™ media comprising titanium dioxide is employed.

An optional inspection port 121 is disposed on a front side of the housing 110 of the air cleaning unit 100. The inspection port 121 is seen in FIGS. 1 and 4. The inspection port 121 provides for safe viewing and inspection of the UV-C lamps 300.

FIG. 11 provides a perspective view of an illustrative odor-reducing medium 400 as may be used in the air cleaning unit 100 of FIG. 1. The odor-reducing medium 400 may be a photocatalytic oxidation (“PCO”) substrate. The medium 400 may in one aspect comprise titanium dioxide that acts as a catalyst for photocatalytic oxidation in the presence of air flow.

In use, once the forced air moves past the germicidal lamps 300, the air moves through the PCO substrate 400 for removal of volatile organic compounds (VOC's), tobacco smoke, chemical fumes and other common household odors. In the illustrated embodiment, the air cleaning unit 100 is equipped with a Quartzel® PCO (photocatalytic oxidation) felt substrate manufactured by Saint-Gobain Quartz of France. The Quartzel® PCO media allows virtually all air passing through the unit to contact the titanium dioxide resulting in increased photocatalysis efficiency. The Quartzel® PCO substrate is cut from 1 meter×1 meter panels and encased in a metal mesh structure that is installed anterior to the UV-C lamps 300. An empty space of approximately 2 inches remains behind the back side of the Quartzel® PCO substrate to reduce static pressure. The Quartzel® PCO substrate is dimensioned to match the cabinet dimensions of the corresponding HVAC unit.

In one embodiment, the Quartzel® PCO substrate has a basis weight of 65 grams per square meter, a thickness of 2 millimeters and a specific surface of 40 m²/g. The Quartzel® fibers utilize the combined action of UV ray and TiO₂ to create OH radicals from water present in the air. The resulting radicals have strong oxidation power capable of decomposing organic compounds and bacteria into CO₂ and H₂O, thus reducing odor. In an alternative embodiment, the air cleaning unit 100 is equipped with a Quartzel® PCO felt substrate having a basis weight of 85 grams per square meter, a thickness of 10 millimeters and a specific surface of 120 m²/g.

One aspect that comes into play when designing an air cleaning unit is static pressure. This is an area of particular concern in residential HVAC applications. Static pressure is an indicator of reduction in airflow. Static pressure has historically been measured with a water gauge. While static pressure may now be measured using an electronic measuring tool, the level of static pressure is still stated in terms of inches in water gauge. (“in. w.g.”).

The components 200, 300, 400 within the air cleaning unit 100 may affect the static pressure in the system. For instance, while higher MERV rated filters used as the air filtering medium 200 will result in a higher level of disinfection, they may also create a higher level of static pressure. Also, different styles of motors (not shown) in the air cleaning unit 100 may change the static pressure in the system. Motor styles typically employed in residential air cleaning units have a static pressure that ranges from about 0.5 in. w.g. to 1.0 in. w.g., depending upon quality and features. A better motor usually has a slightly higher static pressure.

The weight of the odor-reducing medium 400 will also affect the static pressure of the system 100. A medium 400 with a lesser basis weight material will typically have a lower static pressure. For example, a Quartzel™ medium using 65 grams of material per square meter will have a lower static pressure than a Quartzel™ medium having 80 grams of media per square meter.

Also of note, in FIG. 1 an open space 115 is shown at the rear of the Quartzel media 400. (122,115). Eliminating this open space (or bypass) should reduce static pressure due to the less restrictive properties of the 65 gram Quartzel™.

Residential HVAC equipment is available in a variety of sizes and is commonly referred to as “tonnage.” There are 400 cubic feet per meter (“cfm”) per ton. Common HVAC sizes are 1.5, 2.0, 2.5, 3.0, 3.5, 4.0, and 5.0 tons. In addition, air cleaning units such as system 100 may be tested for static pressure per the ASHRAE 52.2 standard at 300 fpm (feet per minute).

A residential HVAC system can typically withstand about 0.50 in. w.g. static pressure. However, not all of this value is available to an air cleaning unit. In this respect, there are other sources of static pressure besides the air cleaning unit. For instance, return ductwork typically creates 0.05 in. w.g. in a residential HVAC system. The supply ductwork typically creates an additional 0.08 in. w.g. In addition, the air conditioner itself (including the motor) typically creates an additional 0.25 in. w.g. This leaves only 0.12 in. w.g. static pressure available for the air cleaning unit. However, the air cleaning unit 100 of the present invention is preferably able to operate within 0.12 in. w.g.

It is noted that the air cleaning components 200, 300, 400 of the system 100 are not limiting of the air cleaning components that may actually be used. In addition to these components, electrostatic filters may be used to reduce the number of suspended particles (e.g., smoke and dust) moving through the chamber 115. Activated charcoal filters for reducing gases and unpleasant odors may also be used.

A plurality of air cleaning units 100 of FIG. 1 may be provided adjacent to one another. In this way, a much larger volume of air may be received and filtered, such as for a commercial HVAC system.

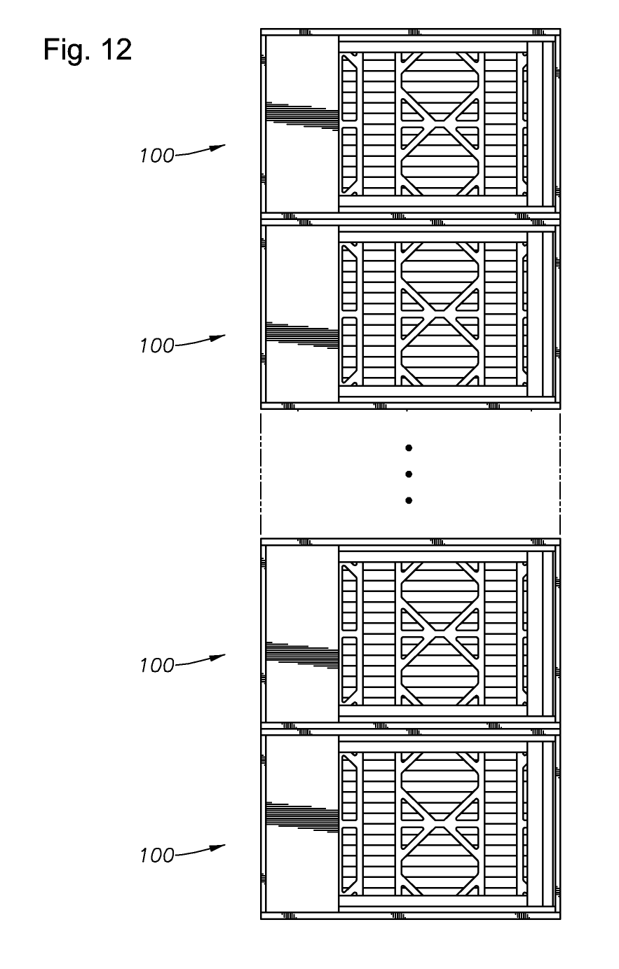

FIG. 12 is a front view of a plurality of air cleaning units 100 assembled to receive and filter air in parallel. The units 100 are placed within an air receiving duct in a commercial building (not shown). In the view of FIG. 12, four air cleaning units 100 are placed in side-by-side arrangement. However, the units 100 may alternatively be placed in a matrix using any number of units 100 as might be needed by an air handling system.

In one aspect, an air cleaning unit is part of an independent, motorized air filtering unit. An example is an air moving unit having filtration, odor removal and disinfection capabilities, but without heating and cooling capabilities.

It should again be understood that the disclosed embodiments are merely exemplary of the inventions, which may be embodied in various forms. Therefore, the details disclosed herein are not to be interpreted as limiting, but merely as the basis for the claims and as a basis for teaching one skilled in the art how to make and/or use the invention.

We claim:

1. An air cleaning unit for an air moving system comprising:
a housing having an air intake and an air outlet;
a particle filtration medium intermediate the air intake and the air outlet;
at least one UV-C lamp intermediate the air intake and the air outlet; and
a VOC and odor-reducing medium, the VOC and odor-reducing medium defining a photocatalytic oxidation filter.

2. The unit of claim 1, wherein the particle filtration medium is fabricated from a material that is resistant to deterioration under exposure to ultraviolet radiation.

3. The unit of claim 2, wherein the particle filtration medium is a MERV rated filter having a MERV rating of about 5 to 11.

4. The unit of claim 3, wherein the air cleaning unit operates within about 0.09 in. w.g. to about 0.31 in. w.g. static pressure.

5. The unit of claim 3, wherein the air cleaning unit is configured to be placed in-line with ductwork for an air handling system.

6. The unit of claim 5, wherein:
the air handling system is a heating, ventilating and air conditioning system; and
the air handling system is designed to handle 1.5 to 5.0 unit tons of air.

7. The unit of claim 5, wherein the unit is part of a motorized air filtering unit without heating and cooling capabilities.

8. The unit of claim 3, wherein the air filtration medium is a five-inch, MERV-11 filter.

9. The unit of claim 1, wherein the housing has at least two sides having double walls.

10. The unit of claim 1, wherein at least two sides of the housing are insulated with a ceramic coating.

11. The unit of claim 10, wherein the photocatalytic oxidation filter:
comprises titanium dioxide; and
is pleated in order to reduce w.g. pressure consumption of the air cleaning unit.

12. The unit of claim 1, further comprising:
a ballast for supporting at least two UV-C lamps; and
at least one socket for connecting the UV-C lamps to the ballast.

13. The unit of claim 12:
wherein the ballast is microprocessor-controlled; and
the air cleaning unit further comprises at least one specular aluminum reflector below each lamp.

14. A method for filtering air that is circulated through ductwork in an air handling system, the method comprising:
providing at least one air cleaning unit, each unit comprising:
a housing having an air intake and an air outlet,
a particle filtration medium intermediate the air intake and air outlet,
at least one UV-C lamp intermediate the air intake and air outlet, and
a photocatalytic oxidation filter for at least partially filtering volatile organic compounds and odors; and
installing the housing of each of the air cleaning units within the air handling system such that each of the at least one units is placed in-line with the ductwork for the air handling system.

15. The method of claim 14, wherein the air handling system is a heating, ventilating and air conditioning system or a furnace.

16. The method of claim 15, wherein:
the at least one air cleaning unit defines a single air cleaning unit; and
the air cleaning unit is installed into the air handling system in either a horizontal position or a vertical position.

17. The method of claim 15, wherein:
the air cleaning unit is installed into the air handling system in a vertical position.

18. The method of claim 16, wherein:
the at least one UV-C lamp is adjacent the air intake;
the air cleaning unit is positioned adjacent fins of a heating, ventilating and air conditioning system; and
the at least one UV-C lamp is oriented perpendicular to the fins of the heating, ventilating and air conditioning system.

19. The method of claim 13, wherein:
the air filtration medium is a MERV rated filter;
the air cleaning unit operates within about 0.12 in. w.g. static pressure

20. The method of claim 15, wherein:
the at least one air cleaning unit defines a plurality of air cleaning units; and
the air cleaning units are installed adjacent to one another so as to receive and filter air as air enters an air handling system.

21. A method for bidding on a residential job specification, comprising:
receiving a request for a bid for the installation of an air cleaning unit from a potential customer; and
responding to the bid by offering an air cleaning unit comprising:
a housing having an air intake and an air outlet,
an air filtration medium intermediate the air intake and the air outlet,
at least one UV-C lamp intermediate the air intake and the air outlet, and
a photocatalytic oxidation filter; and
wherein the air cleaning unit has about a 0.09 in. w.g. to about 0.31 in. w.g. static pressure.

22. The method of claim 21, further comprising:
installing the air cleaning unit into a heating, ventilating and air conditioning system for the potential customer.
